(19) United States
(12) Patent Application Publication
(10) Pub. No.: US 2026/0132428 A1
(43) Pub. Date: May 14, 2026

(54) METHOD FOR CONVERTING LIGNOCELLULOSIC BIOMASS

(71) Applicant: IFP ENERGIES NOUVELLES, Rueil-Malmaison (FR)

(72) Inventors: Caroline AYMARD, Rueil-Malmaison Cedex (FR); Matthieu ARAGONES, Rueil-Malmaison Cedex (FR); Sophie COUDERC, Rueil-Malmaison Cedex (FR); Gilles FERSCHNEIDER, Rueil-Malmaison Cedex (FR); Romain ROUSSET, Rueil-Malmaison Cedex (FR)

(73) Assignee: IFP ENERGIES NOUVELLES, Rueil-Malmaison (FR)

(21) Appl. No.: 19/119,629

(22) PCT Filed: Sep. 28, 2023
(86) PCT No.: PCT/EP2023/076939

§ 371 Date: Apr. 9, 2025

(30) Foreign Application Priority Data
Oct. 10, 2022 (FR) FR2210337

(51) Int. Cl. C12P 7/06; C12P 19/02

(52) U.S. Cl. CPC C12P 7/06; C12P 19/02; C12P 2201/00

(57) ABSTRACT

The present invention relates to a process for converting lignocellulosic biomass by contacting, in aqueous phase, pretreated lignocellulosic biomass with at least one biocatalyst (3) in a first reactor (1) containing a reaction medium comprising said pretreated lignocellulosic biomass (2) in aqueous phase and said biocatalyst, said process comprising:
(a) a first step of liquefaction by addition of said pretreated lignocellulosic biomass and at least one biocatalyst to said reactor without withdrawing all or part of said reaction medium from said reactor,
and then (b) a second step of continuous liquefaction, with continuous withdrawal, from said first reactor, of a portion of said reaction medium, addition of at least one biocatalyst, and continuous addition of pretreated lignocellulosic biomass.

### TECHNICAL FIELD

The invention relates to a process for converting lignocellulosic biomass to produce “second-generation” (2G) sugars (or sugary liquors).

These sugars may be used to produce other products via a biochemical and/or catalytic pathway (for example alcohols such as ethanol, butanol or other molecules, for example xylitol, solvents such as acetone, etc.).

This process generally comprises a pretreatment of the biomass, which may comprise, for example, impregnation with a liquor containing a chemical catalyst, such as an acid, a base or an oxidizing compound, followed by cooking of the impregnated biomass, which cooking is optionally coupled with a steam explosion. Once pretreated, the biomass is then converted into sugars by enzymatic hydrolysis under the action generally of an enzymatic cocktail comprising at least one cellulolytic enzyme. The sugars thus formed can be fermented into alcohol under the action of yeasts or bacteria, either in a fermentation step separate from the enzymatic hydrolysis step, or simultaneously with the enzymatic hydrolysis. In the latter case, reference is made to SSCF, an acronym for “simultaneous saccharification and co-fermentation”, when the sugars fermented are a mixture of C5 and C6 sugars obtained by enzymatic hydrolysis (i.e. having 5 or 6 carbons), or SSF, for “simultaneous saccharification and fermentation”, when only C6 sugars are fermented.

### PRIOR ART

The invention is concerned more particularly with the enzymatic hydrolysis of pretreated biomass, optionally combined with SSF- or SSCF-type fermentation. The pretreated biomass is the substrate of the enzymatic hydrolysis reaction. The residence time of the substrate is defined as the average residence time of the latter under the reaction conditions. The cycle time is considered here to be the time between two cleaning operations.

The enzymatic hydrolysis can be carried out in various ways. Conventionally, enzymatic hydrolysis can be performed in batch mode, fed-batch mode or continuously.

Performance in batch mode can be summarized as follows: the substrate is added to the conversion reactor at the start of the cycle, and then left in contact with the biocatalysts for a reaction time necessary to achieve the target conversion, before the reaction medium is completely emptied. In this configuration, the residence time of the substrate is homogeneous, since all of the substrate is added and removed at the same time. The cycle time of the reactor consists of the time for preparation of the reactor, the reaction time (the residence time of the substrate) and the emptying and cleaning time.

Performance in continuous mode can be summarized as follows: the substrate is added and a fraction of the reaction medium withdrawn over time. These additions and withdrawals can be done in real-continuous mode, or in pseudo-continuous mode: that is to say that the time between the withdrawals/additions is much quicker than the average residence time of the substrate. In this configuration, the cycle time of the reactor can be greatly extended. The average residence time of the substrate can be defined as the ratio between the volume of the reaction medium in the reactor and the average hourly flow rate of substrate added. This configuration allows a better use of the available reactor volume; however, it has the disadvantage of exhibiting heterogeneity in the residence time of the substrate and biocatalysts in the reactor: this is because the withdrawal carried out is a withdrawal of the reaction medium and therefore a portion of the substrate and/or of the biocatalysts added will be withdrawn during the withdrawal without having achieved the average residence time. Conversely, another portion will remain in the reactor for longer than the average residence time. Consequently, in the case of reactions involving biocatalysts, that is to say microorganisms such as yeasts or bacteria, there is a greater risk of performance drifts associated either with the appearance of contamination or to a change in the biocatalyst (for example, loss of a genetic modification of interest) as a result of the prolonged residence time for a portion of the inventory.

Performance in fed-batch mode is quite similar to performance in batch mode: a portion of the substrate is gradually added to the reactor while the desired reaction(s) have started in the reactor, and then the entire inventory of the reactor is emptied at the end of the cycle. This type of performance is conventional for bioprocesses and makes it possible to circumvent the typical limitations of bioconversions: the performance in fed-batch mode is, for example, carried out when the medium exhibits an excessively high content of toxic molecules, or when the initial rheology of the medium is problematic.

It is thus possible, as described in patent EP-3 461 902, to perform the enzymatic hydrolysis in batch mode with a sequential feed of pretreated biomass (referred to as “fed-batch” feed), where the sequential addition to the hydrolysis reactor is carried out in a manner increasingly spaced-apart in time, so as to obtain a predetermined final solids content, without withdrawal during the hydrolysis.

This type of feed is interesting in that it makes it possible to obtain an improved yield of conversion to sugar and that it makes it possible to work with a high solids content, which leads to high concentrations of product of interest in the medium, and also because it makes it possible to better control variations in viscosity of the reaction medium: as the hydrolysis reaction progresses, the reaction medium becomes less and less viscous, and pretreated biomass can thus be added so as to raise the solids content of the reaction medium again.

However, there are limits to this solution, similar to the limits of performance in batch mode, with the regular necessity for emptying and cleaning times. These times cannot be shortened and reduce the utilization rate of the vessel, in particular when the reaction time is less than 24 hours, which adversely affects productivity.

As described, for example, in patent WO 2013/088001, it is also known to break the enzymatic hydrolysis down into two steps, each performed in a specific reactor:
a step referred to as a liquefaction step, which corresponds to the start of hydrolysis, during which the reaction medium is viscous and requires a complex stirring system and a large amount of stirring energy for the reactor. On the other hand, the residence time of the biomass is generally short, which makes it possible to limit the volume of the reactors to be installed with such a stirring system.
a following step, which corresponds to saccharification in the case of only continuing hydrolysis, or to SSF or SSCF in the case where microorganisms are introduced to ferment the sugars while at the same time hydrolyzing or hydrolyzing and fermenting, in another reactor. This step requires simpler stirring, and less energy, but a longer residence time and therefore a larger reactor volume.

According to the teaching of this patent, a rheological characteristic of the reaction medium is monitored during the first liquefaction step, so as to accordingly adjust the rates of feeding the reactor with pretreated biomass/water/enzymes/other inputs (chemicals, acid or base for example), and thus optimize the liquefaction. This solution is of interest in that it makes it possible to have an efficient performance of the first liquefaction step at least, regardless of the nature of the biomass, without having to characterize it. On the other hand, in industrial production, it requires a substantial number of reactors overequipped with stirring means and with a relatively small volume for performing the liquefaction. Operation in batch mode also adversely affects the productivity as a result of the frequency of the emptying, cleaning and filling operations.

The aim of the invention is consequently to overcome the drawbacks of the prior solutions. It aims to improve the enzymatic hydrolysis processes, in particular to reduce and simplify the necessary equipment and/or to reduce the energy consumption of said equipment, without degrading—or even while increasing—the biomass conversion yields.

### SUMMARY OF THE INVENTION

A first subject of the invention is a process for converting lignocellulosic biomass by contacting, in aqueous phase, pretreated lignocellulosic biomass with at least one biocatalyst in a first reactor containing a reaction medium comprising said biomass in aqueous phase and said biocatalyst, said process comprising:
(a) a first step of liquefaction by addition of said pretreated lignocellulosic biomass and at least one biocatalyst to said reactor without withdrawing all or part of said reaction medium from said reactor, —and then (b) a second step of continuous liquefaction, with continuous withdrawal, from said first reactor, of a portion of said reaction medium, addition of at least one biocatalyst, and continuous addition of pretreated lignocellulosic biomass.
   (This continuous addition makes it possible to maintain the reaction volume in the reactor at a given level in the reactor).

The biomass targeted by the invention is lignocellulosic in nature with very varied substrates comprising ligneous substrates such as various woods (hardwoods and softwoods), coproducts derived from agriculture (wheat straw, corn cobs, etc.) or from other agrifood or paper industries, lignocellulosic waste, etc.

The term “pretreated” in relation to the biomass is understood in its usual sense in the field of the treatment of lignocellulosic biomasses. This generally involves impregnation with an acidic, basic or oxidizing liquor or simply with water, followed by an optional cooking, in particular combined with a steam explosion. For further details on this prior operation, reference may be made for example to patents FR 3054141, FR 3075202 and FR 3075203.

The reaction medium is in aqueous phase: The water may originate from the biomass itself (which contains water naturally and/or which has been impregnated with aqueous solution(s) prior to the treatment of the invention). The water may also originate from a specific supply of water.

The term “biocatalyst” is understood to mean, as detailed below, an enzyme or a mixture of enzymes and/or a type of microorganism or multiple types of microorganisms, in particular of the bacterium or yeast type. The biocatalyst(s) may be added, like the biomass, continuously or discontinuously, or in one go.

For the purposes of the present invention, the term “continuous” withdrawal and “continuous” feed includes a continuous withdrawal and feed in the strict sense, or a pseudo-continuous withdrawal and feed. The term “pseudo-continuous” is understood to cover the fact that the withdrawal and/or feed of substrate may be done sequentially. By way of illustration of this mode of transfer: x kg of medium can be withdrawn for a duration of y minutes, then x kg of substrate and other inputs necessary for the reaction (biocatalysts, chemicals, acid or base for example) are added for a duration of z minutes, and so on for the entire duration of the step, for 20 to 24 hours for example. The duration of withdrawal and of addition may be different or similar. The amounts of substrate and all other inputs added are the same as those withdrawn so as to keep the volume in the reactor the same.

The invention therefore proposes to proceed with the conversion of pretreated biomass by breaking the conversion down into two stages, the first of which, the liquefaction, has been modified compared to the modes of operation already known for liquefaction by adding to a liquefaction step, preferably via sequential addition (“fed-batch” mode), a following step that is performed continuously. In this continuous step, the withdrawal from the reactor and the feed of pretreated biomass are continuous, or pseudo-continuous. The withdrawal flow rate and the feed of pretreated biomass will be adapted throughout this step in order to regulate, in particular, the rheology of the reaction medium in the reactor.

The process according to the invention therefore provides for a liquefaction step (a), in which the components of the reaction medium are added to the reactor/brought into contact (biomass, water, biocatalyst) for an initiation of the liquefaction, without withdrawal. Then liquefaction then continues with step (b) with continuous withdrawal.

And, with this additional step of continuous liquefaction, the invention imparts an enormous industrial advantage on the conversion process as a whole (including a step of enzymatic hydrolysis proper after the liquefaction): it makes it possible to maximize the utilization rate of the reactors dedicated to the liquefaction.

The invention thus makes it possible, for a given production of converted biomass, to limit the number of liquefaction reactors to be put into use (or to increase the production of converted biomass for a given number of liquefaction reactors to be put into use) by limiting the frequency of the non-productive feeding, emptying and cleaning phases.

This point is all the more advantageous, in terms of return on industrial investments, since liquefaction reactors must in general, as already mentioned, be equipped with complex stirring systems the operation of which is highly energy-consuming.

It was also found that, with this mode of liquefaction, the frequency of cleaning of the liquefaction reactors could be reduced without negative impact.

It was additionally also found that with the liquefaction according to the invention, continued by the conversion stage proper in another reactor (enzymatic hydrolysis or enzymatic hydrolysis and simultaneous fermentation SSF or SSCF), the same yields of conversion to sugar or alcohol were obtained.

It should also be noted that, with the liquefaction performed according to the invention, no problems are encountered as regards the control of the viscosity of the reaction medium, and no difficulties are encountered either in withdrawing/emptying the liquefaction reactor or in filling the next reactor allowing the conversion reaction to be continued.

Advantageously, the pH of the aqueous phase may be regulated by controlled addition to the first reactor of at least one acidic and/or basic compound in at least one of the two liquefaction steps (a) and (b). Thus, generally, if the biomass was pretreated with an acidic liquor, the pH will tend to be adjusted to and then maintained at a set value by the controlled addition of base, and, if it was pretreated with a basic liquor, the pH will tend to be adjusted to the set value by the controlled addition of acid. The pH may also be regulated before the liquefaction.

During the first liquefaction step (a), the additions of said pretreated lignocellulosic biomass to the first reactor may be performed according to a fixed or variable frequency, and in fixed or variable amounts. The biomass may also be added to the reactor in one go. The same goes for the biocatalyst(s), and water (when make-up water is added should the biomass not contain enough). The water may be added separately from the biomass, or the biomass may have already been brought into contact with all or some of the water before addition to the reactor. According to one embodiment, all of the components of the reaction medium, and therefore the water, the pretreated biomass and the biocatalyst(s), are added in one go, optionally at the same time, and preferably at the very start of step (a).

During the first liquefaction step (a), the additions of said pretreated lignocellulosic biomass to the first reactor may be performed according to increasingly spaced-apart intervals of time, as described for example in abovementioned patent EP 3 461 902, and preferably with fixed amounts of biomass.

During the first liquefaction step (a), the biocatalyst or at least one of the biocatalysts may also be added sequentially to the reaction medium, either with the same frequency and the same spacing as the pretreated biomass or with a different frequency and spacing.

Alternatively, the biocatalyst(s) may be added in one go, during, and particularly at the very start of, liquefaction step (a).

During the first liquefaction step (a), the additions of the pretreated lignocellulosic biomass to the first reactor may be carried out continuously or sequentially.

The second liquefaction step (b) is preferably performed with a constant volume of the reaction medium contained in the first reactor. Since a portion of the reaction medium is continuously withdrawn, pretreated biomass and the necessary inputs are therefore gradually added such that the volume of the reaction medium remains substantially unchanged and “fresh” pretreated biomass can be liquefied throughout this step (b). These additions are continuous according to a given frequency, or controlled via monitoring of a given physicochemical, rheological or operational characteristic of the reaction medium.

During the second liquefaction step (b), pretreated lignocellulosic biomass and at least one other compound referred to as “input”, at least one of which is chosen from one of the following compounds: water, acidic compound, basic compound, biocatalyst(s), will therefore be added over time, and the latter compound may be added with the biomass, or in several goes but not at the same time as the biomass, or in one go at the start of the step. Preferably, the biocatalyst(s) are added at the same time/with the biomass.

These inputs, and also those present at the initiation of the liquefaction, may also contain other compounds, additives for example of antifoam agent or antibacterial agent type, or also nutrients (in the case of SSF or SSCF in particular, for the microorganisms used for the fermentation).

The rheology of the reaction medium may be adjusted during the second liquefaction step (b) according to at least one operating condition from among: the residence time of the pretreated lignocellulosic biomass in the first reactor, the amount and/or the frequency of the additions of pretreated lignocellulosic biomass and input(s) at least one of which is chosen from one of the following compounds: water, acidic compound, basic compound, biocatalyst(s).

The rheology of the reaction medium may be adjusted during the second liquefaction step (b) so that it is identical to or less severe than the rheology of the reaction medium at the end of the first liquefaction step (a). The more “severe” the rheology of a reaction medium, the more, in particular, it will be necessary to equip the reactor with high-performance stirring means and/or the more energy will have to be consumed to operate them.

It is thus sought to maintain within the liquefaction reactor an appropriate rheology in the continuous liquefaction according to the invention, so that the reaction medium remains at viscosity conditions allowing it to be stirred in the reactor and withdrawn to another reactor in a manner compatible with industrial-scale production.

The rheology of the reaction medium may be monitored by monitoring the viscosity of the reaction medium or the mechanical torque on the shaft of a stirring system fitted to the first reactor or the electric power consumed by the motor driving said stirring system.

The first liquefaction step (a) preferably has a duration of between 1 and 48 hours, more preferentially still between 2 and 24 hours, and in particular between 5 and 12 hours.

The second liquefaction step (b) has a duration preferably of between 1 and 170 hours, in particular between 10 and 72 h, in particular between 15 and 30 hours or between 20 and 28 hours.

During the second liquefaction step (b), the residence time of the pretreated biomass in the first reactor is preferably greater than or equal to 4 hours, in particular greater than or equal to 5 hours, for example between 5 hours and 14 hours.

During the second liquefaction step (b), a portion of the reaction medium may advantageously be continuously withdrawn from the first reactor and sent to a second reactor in which, in a conversion step (c), the conversion of the biomass contained in the withdrawn reaction medium is continued in the presence of at least one biocatalyst.

Biocatalysts are already contained in the reaction medium transferred from one reactor to the other, but biocatalysts different from or identical to those already introduced into the first reactor may be specifically added to the second reactor.

Thus, when aiming for conversion of the biomass into alcohol by SSF or SSCF:
according to a first embodiment, it is possible to add all of the biocatalysts (enzymes and microorganisms) starting from the liquefaction in the first reactor (and therefore not to add biocatalyst in step c) in the second reactor), —and, according to another embodiment, it is possible to add the enzymes during the liquefaction in the first reactor, and then to add the microorganisms, and optionally additional enzymes, in step c) in the second reactor.

At the end of the second liquefaction step (b), all of the reaction medium is advantageously transferred from the first reactor to a second reactor in which, in a conversion step (c), the conversion of the biomass contained in the transferred reaction medium is continued in the presence of at least one biocatalyst.

Advantageously, the duration of the second liquefaction step (b) is less than or equal to the duration of the conversion step (c).

Step (c) in the second reactor mentioned above may therefore be performed in fed-batch mode, preferably with a feed duration of between 1 hour and 50 hours, and preferentially between 10 hours and 40 hours, followed by a duration of operation in batch mode. The total duration of step (c), in fed-batch mode and then in batch mode, is preferably between 10 and 170 hours, in particular between 70 and 140 hours. “Batch mode” is to be understood in its usual meaning, namely that there is no withdrawal from the reactor throughout the duration of the conversion performed in this reactor.

The coupling of steps (a) and (b) in a dedicated liquefaction reactor and (c) in another reactor makes it possible to benefit from the combined advantages of previous batch, fed-batch and continuous implementations: —the liquefaction reactor is operated according to steps (a) and then (b) which make it possible to maximize the use of this reactor for containing reaction medium, and to reduce the time allotted for emptying and cleaning during a cycle—the reactor performing step (c) ends in batch mode, which makes it possible to maximize the degrees of conversion achieved, and to control the maximum residence time of the biocatalysts, thus avoiding the drifts mentioned above.

Advantageously, the second reactor in which step (c) takes place has a volume greater than that of the first reactor in which steps (a) and (b) take place. Preferably, the volume of the second reactor is greater than 100% of the volume of the first reactor, preferably greater than 120%, preferentially greater than 200% and more preferentially still greater than 300%. The second reactor in which step (c) takes place may be fed by a plurality of reactors in which steps (a) and (b) take place.

It is also possible to use a plurality of reactors to perform step (c) which are smaller in size and operate in particular in series.

The biocatalyst added for at least one of the first liquefaction (a), second liquefaction (b) and conversion (c) steps, and especially all of these steps, advantageously comprises at least one enzyme for converting the pretreated biomass at least partially into sugar(s) by enzymatic hydrolysis, and optionally at least one microorganism, of yeast or bacterium type, for converting all or some of this/these sugar(s) into alcohol(s) by fermentation.

According to a variant, the biocatalyst used for each of the first liquefaction (a) and second liquefaction (b) steps may comprise at least one enzyme for converting the pretreated biomass at least partially into sugar(s) by enzymatic hydrolysis, and the biocatalyst added in conversion step (c) may comprise a mixture of enzyme(s) and yeast(s) (or other microorganism) or just at least one yeast for converting all or some of the sugar(s) transformed into alcohol(s) by fermentation.

According to a variant, the biocatalyst used for fermenting all or some of the sugar(s) into alcohol(s) or products of interest is a bacterium, for example Clostridium, such as Clostridium acetobutylicum. It may be added to the process in the same way as the yeasts mentioned above.

Specifically, the present invention is aimed at converting pretreated biomass in order to produce sugars by enzymatic hydrolysis, generally using a cocktail of enzymes comprising at least cellulolytic enzymes. The cellulolytic enzymes are, for example, cellulases, endoglucanases, beta-glucosidases. The enzymatic cocktail may also comprise hemicellulolytic enzymes (hemicellulases). The enzymes may be produced by bacteria or fungi. Preferably, the enzymes are produced by a fungus, for example Trichoderma reseii. The liquefaction according to the invention will therefore use this kind of cocktail, as will the conversion reaction which will continue in a reactor other than the liquefaction reactor. The sugars in question may be utilized as such or after transformation.

The present invention is also aimed at the production of alcohols by fermentation from these sugars, according to two main types of process: —either saccharification and fermentation are performed at the same time, this is known as the SSF or SSCF process. In this case, appropriate microorganisms (yeast, bacteria, as seen above) are added to the enzymes. The microorganisms may thus be added starting from the liquefaction, —or the fermentation takes place after saccharification, in a reactor dedicated to the fermentation and fed with appropriate microorganisms. In this case, a single type of biocatalyst is added to each step (enzymes for the hydrolysis, and then microorganism for the fermentation).

It should be noted that, for the performance of the fermentation, the biocatalyst is a microorganism, which may be a yeast or a bacterium, even if, in the present text, it is possible only yeasts are mentioned for reasons of conciseness.

According to one embodiment, the process according to the invention may comprise the following steps: —an initial step (a0) of filling the first reactor with a first provision in said reactor of pretreated lignocellulosic biomass, biocatalyst(s), water and optionally acidic and/or basic compounds, —a first step (a) of liquefaction in the first reactor by addition of said pretreated lignocellulosic biomass to said reactor without withdrawal, with the optional addition also of biocatalyst(s), —then a second step (b) of continuous liquefaction in the first reactor, with continuous withdrawal, from the first reactor, of a portion of the reaction medium and transfer to a second reactor of said portion of the withdrawn reaction medium and continuous addition of pretreated lignocellulosic biomass, and optionally of water and/or biocatalyst(s), and/or acidic and/or basic compounds, —then an optional step (b1) of homogenization of the reaction medium in the first reactor, —then a step (b2) of transfer of all of the reaction medium from the first reactor to the second reactor, in which conversion step (c) is performed preferably in batch mode, —and a step (b3) of cleaning the first reactor, in particular with the aid of an aqueous and preferably acidic or basic solution.

The additions of said pretreated lignocellulosic biomass to the first step a) may or may not be sequential.

The additions of the biocatalysts to the first step a) may or may not be sequential.

The operating conditions of the first liquefaction (a) in enzymatic-hydrolysis-only configuration (i.e. when targeting a conversion of the biomass into oligomeric or monomeric sugars) are preferably: —a temperature of between 25 and 80° C., preferably of between 4° and 60° C., and more preferentially still between 45° C. and 55° C.,
a pH of between 3 and 7, preferably of between 4 and 6 and more preferentially still of between 4.8 and 5.5.

The operating conditions of the first liquefaction (a) in simultaneous enzymatic hydrolysis and fermentation configuration (SSF or SSCF), i.e. when targeting the conversion of the biomass into alcohol by hydrolysis and fermentation, are: —a temperature of between 25 and 80° C., preferably of between 3° and 50° C. and more preferentially still between 30° C. and 35° C., —a pH of between 3 and 7, preferably of between 4 and 6 and more preferentially still of between 5.0 and 5.5.

The operating conditions (temperature and pH) of the second liquefaction (b) are preferably identical to those of the first liquefaction (a).

The operating conditions (temperature and pH) of the conversion step (c) may be identical to or different from those of the first liquefaction (a) and the second liquefaction (b). They will, for example, be different if the biocatalysts introduced during this step are different from those introduced during the first liquefaction (a) and the second liquefaction (b).

Preferably, the solids content SC of the pretreated lignocellulosic biomass used in the process according to the invention is at least 2% by weight, in particular at least 5% by weight, or at least 10% by weight. The biomass used in the process according to the invention contains at least 10 g of cellulose per 100 g of solids, in particular at least 20 g of cellulose per 100 g of solids.

### LIST OF THE FIGURES

FIG. 1 shows the different stages of a step of liquefaction of pretreated biomass according to the prior art.

FIG. 2 shows the different stages of a step of liquefaction of pretreated biomass according to an embodiment of the invention.

FIG. 3 is a graph which shows the evolution of the ethanol and xylose concentrations over the course of an SSCF process with liquefaction according to the prior art and with liquefaction according to the invention as a function of time. On the y axis the concentrations are in g/kg of reaction medium, and on the x axis the time is expressed in hours.

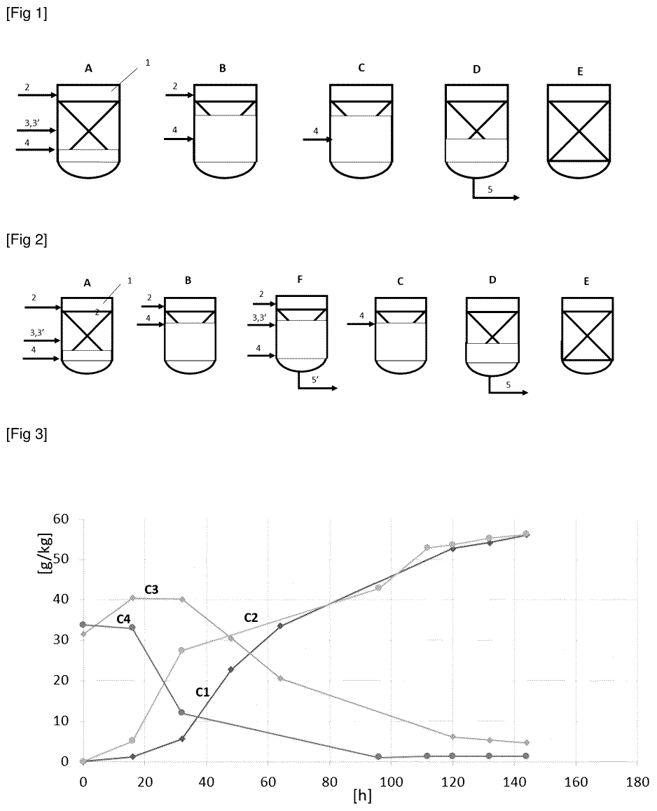

The figures, and more precisely FIGS. 1 and 2, are highly schematic and are not true to scale. Identical references refer to the same flows/devices from one figure to another.

### DESCRIPTION OF THE EMBODIMENTS

The aim of the invention is to improve the procedure for the liquefaction of pretreated lignocellulosic biomass. Liquefaction is to be understood as a step of initiation of the conversion of the biomass under the effect of biocatalysts. This is a conversion by enzymatic hydrolysis (and/then optional fermentation). This liquefaction is sometimes referred to as “pre-hydrolysis”.

One known protocol for performing the enzymatic hydrolysis alone or the enzymatic hydrolysis and simultaneous fermentation of biomass or lignocellulosic waste is a first liquefaction step of fed-batch type (sequential addition of biomass and no withdrawal throughout the step) in a reactor specially dimensioned/designed for this purpose, and then transfer to a more standard reactor for continuing, in a second step, the enzymatic hydrolysis alone or the simultaneous enzymatic hydrolysis and fermentation in batch mode.

Reference may be made, for example, to the abovementioned patent WO 2013/088001 for the description of this type of protocol.

The protocol according to a preferred embodiment of the invention proposes a liquefaction with a first step of fed-batch type of lignocellulosic biomass in a dedicated reactor (relatively restricted useful volume with high-performance stirring equipment), which is followed in this same reactor by a step of continuous operation, with: —a continuous or pseudo-continuous transfer to a more standard reactor (the useful volume of which can be much greater and the stirring equipment of which is simpler than the first reactor) in order to continue the enzymatic hydrolysis alone or the simultaneous enzymatic hydrolysis and fermentation in batch mode, —and a continuous or pseudo-continuous feed of lignocellulosic substrate and various inputs/biocatalysts to the first reactor.

By way of this new protocol, it has been shown that the utilization rate of the liquefaction reactors is maximized, making it possible, for a given production, to limit the number of liquefaction reactors to be put into use, for the same productivity in enzymatic hydrolysis (or in SSF or SSCF).

The invention relates to the implementation of enzymatic hydrolysis for the production of sugars or of enzymatic hydrolysis and simultaneous fermentation for the production of alcohol from lignocellulosic biomass/waste.

The feedstock treated by the process according to the invention is a pretreated lignocellulosic biomass. The pretreatment of the lignocellulosic biomass enables the cellulose to be made accessible and reactive to the enzymes and consists in contacting the lignocellulosic biomass with a solvent and optionally a catalyst (generally combined in a liquor) at a given temperature and pressure for a given residence time. Any type of pretreatment may be applied in order to obtain the pretreated lignocellulosic substrate.

The pretreated biomass may also be subjected to washing with water (resuspending the pretreated biomass with water or a mixing fluid, solid/liquid filtration, washing the solid fraction with water and then solid/liquid filtration) after pretreatment thereof and before the initiation of the liquefaction according to the invention.

During the step of enzymatic hydrolysis or SSF or SSCF (which includes the liquefaction steps according to the invention exemplified below), the pretreated lignocellulosic substrate is mixed with a liquid solution containing the enzymes (and optionally the microorganisms such as yeasts or bacteria). The objective is to obtain a high concentration of ethanol (or sugars if fermentation is not performed). The fermentation/enzymatic hydrolysis step should be carried out at relatively high concentrations of pretreated lignocellulosic substrate, i.e. with a high solids content, in order to reduce the economic and energetic costs of the process if the product of interest needs to be concentrated.

This solids content (acronym “SC”) denotes the solids content measured according to the standard ASTM E1756-08 (2015) “Standard Test Method for Determination of Total Solids in Biomass”. (The concentration of pretreated lignocellulosic substrate in the medium can be expressed as percentage by weight of solids).

Intimate mixing of the pretreated lignocellulosic substrate with said liquid solution containing the enzymes (and optionally the yeasts) proves to be difficult when the solids contents are high. Specifically, the start of the enzymatic hydrolysis with a high solids content poses problems in particular of mixing and homogenization. The reaction medium is very pasty and viscous.

To address this problem, the existing solutions are: —to equip the fermentation (or enzymatic hydrolysis) reactors with a specific complex stirrer in order to guarantee homogenization of the reaction medium. —to carry out a gradual feed of substrate, referred to as “fed-batch” feed, into the reactor, without withdrawing the reaction medium. As the reaction progresses, the mixture becomes less and less viscous and fresh substrate can be added so as to increase the amount of substrate in the medium.
to conduct the fermentation (or enzymatic hydrolysis) in two steps: A first step referred to as liquefaction, which makes it possible to reduce the viscosity of the medium. This step actually corresponds to the first hours of the enzymatic hydrolysis (or SSF/SSCF); the cellulose (which is insoluble in the medium) is converted into oligomeric or monomeric sugars that are soluble in the medium. It ends when the viscosity has been reduced to a value that allows transfer to a tank equipped with a standard stirrer for continuation of the enzymatic hydrolysis. A second step corresponding to the continuation of the enzymatic hydrolysis (or SSF/SSCF): the liquefied biomass obtained from the liquefaction step is transferred to fermentation (or hydrolysis) reactors in which the conversion of the cellulose and residual hemicelluloses into sugars and then the conversion of the sugars into ethanol continues.

The invention is interested in this latter approach and aims to improve it.

### Prior/Comparative Embodiment

The step of liquefaction of the pretreated substrate is carried out in “fed-batch” mode, that is to say with the addition of the substrate gradually to the mixture of water+biocatalysts+base. (Specifically considered in this case is the non-limiting example of a pretreatment with impregnation by an acidic liquor, hence the addition of a base in order to increase the pH of the reaction medium).

Due to the rheology of the medium, the liquefaction reactor is first charged with a portion of the substrate to be treated and all of the water, the pH and the temperature are adjusted to the required setpoints, and then some or all of the enzymes (and possibly yeasts) are added. A fed-batch (sequential feed) mode is then carried out with the rest of the pretreated substrate in order to increase the solids content. A fed-batch mode for the enzymes (and yeasts) may also be carried out. The rheology of the solid suspension requires a particular implementation in order to guarantee good stirring to carry out the reaction in a medium that is as concentrated as possible in pretreated lignocellulosic substrate. To alleviate a portion of this constraint, particular stirrer technology may be fitted to the liquefaction reactor.

Helical-type stirrers are generally the most suitable, even if they are complex and limited in size for mechanical design reasons.

Various parameters make it possible to to define the operating conditions and establish the fed-batch strategy. The ranges indicated below are examples: —the solids content (SC) of the pretreated biomass (2% to 60% by weight)—the cellulose content of the substrate
the dose of enzymes relative to the cellulose (5 to 100 mg/g of cellulose)
the rate of inoculation with yeasts (0.1 to 3 g/kg of medium),
the solids content (SC) of the initial mixture (2% to 60% by weight)
the solids content (SC) of the final mixture (2% to 60% by weight)
the total volume and/or mass of the final mixture in the reactor
the number of fed-batch additions
the duration of the fed-batch additions (0 to 48 h)
the duration of the liquefaction (1 to 48 h)

The description of the prior fed-batch liquefaction protocol is as follows:
1—Preparation of the initial mixture in the liquefaction reactor: a certain amount of pretreated biomass is mixed with a certain amount of water in order to achieve the desired solids content (SC). The stirring is started in order to homogenize the mixture and adjust the pH and temperature. An antibacterial agent, for example of the chloramphenicol type or that sold under the trade name VitaHop by the company BetaTec, may be added to the reaction medium.
2—Regulation of the pH with the addition of a basic solution, for example NH₄OH or KOH or NaOH if the pretreated substrate was produced under acidic conditions (for example impregnation with sulfuric acid and then steam explosion) or with the addition of an acidic solution if the pretreated substrate was produced under basic conditions. The regulation of the pH may be maintained during the following phases of the protocol.
3—Injection of the enzymes (and of the yeasts) and then starting of the fed-batch liquefaction: once the initial mixture is suitably homogeneous, a certain amount of enzymes (and of yeasts) is introduced into the reactor. This injection makes it possible to achieve the chosen dose of biocatalysts. After the injection of the biocatalysts, the fed-batch liquefaction starts.
4—Fed-batch-additions of pretreated biomass: after a certain predetermined time, the medium is already much less viscous and the first addition of pretreated biomass can take place. Depending on the strategy for adding the enzymes, additional enzymes may be added at this time. The mass of enzymes introduced corresponds to the addition of pretreated biomass. It is possible to choose to add an amount of enzymes according to the amount of pretreated biomass added in each addition of biomass, or else to add all or the major portion of the amount of enzymes needed as soon as the start of the fed-batch operation. The additions follow one another regularly until a mixture with the desired final mass is achieved. Generally, the mass and rate of each addition are constant and regular. As seen above, it is also possible to increasingly space apart the biomass additions. In general, the objective is to aim for a target enzyme dose expressed in grams per kilogram of cellulose.
5—End of the liquefaction: after the phase of the pretreated biomass additions, the reaction continues to constant final volume. The reaction and the fall in viscosity continue up to the moment that the viscosity is judged to be sufficiently low to be able to transfer the medium to the enzymatic hydrolysis (or SSF or SSCF) reactor equipped with a standard stirrer.
6—After transfer of the reaction medium to an enzymatic hydrolysis (or SSF or SSCF) reactor corresponding to a conventional stirred tank, the liquefaction reactor is cleaned to limit the risks of contamination.

The operating conditions of the liquefaction in enzymatic-hydrolysis-only configuration (i.e. when targeting a conversion of the biomass into oligomeric or monomeric sugars) are:
a temperature of between 25 and 80° C., preferably of between 4° and 60° C., and more preferentially still between 45° C. and 55° C.,
a pH of between 3 and 7, preferably of between 4 and 6 and more preferentially still of between 4.8 and 5.5.

The operating conditions of the liquefaction in simultaneous enzymatic hydrolysis and fermentation configuration (SSF or SSCF), i.e. when targeting the conversion of the biomass into alcohol by hydrolysis and fermentation, are:
a temperature of between 25 and 80° C., preferably of between 3° and 50° C., and more preferentially still between 30° C. and 35° C.,
a pH of between 3 and 7, preferably of between 4 and 6 and more preferentially still of between 5.0 and 5.5.

The duration of the liquefaction is between 1 hour and 48 hours, in particular between 2 hours and 24 hours, in particular between 5 hours and 12 hours.

FIG. 1 shows the different cycle phases/times A to E of the liquefaction, representing the liquefaction reactor 1 at each of these phases:
A: Filling of the liquefaction reactor 1 with pretreated biomass 2, water 3′ and biocatalyst(s) 3, and a basic compound 4, and regulation of the temperature and pH of the reaction medium in the reactor: duration between 2 hours and 4 hours
B: Fed-batch operation with pretreated biomass, with sequential addition of pretreated biomass 2 and optionally of basic compound 4 to regulate the pH: duration from 2 hours to 10 hours
C: Homogenization (optional): from 1 hour to 2 hours
D: Transfer of the reaction medium 5 from the reactor 1 to a downstream reactor (not shown): duration between 2 h and 4 h
E: Cleaning of the liquefaction reactor 1: between 2 hours and 4 hours

The enzymatic hydrolysis (or SSF or SSCF) is performed in batch mode. The operating conditions of pH and temperature are generally identical to the liquefaction. The duration of the enzymatic hydrolysis (or SSF or SSCF) is between 10 hours and 170 hours, preferably between 48 hours and 140 hours. After emptying the enzymatic hydrolysis (or SSF or SSCF) reactor, the reactor is cleaned to limit the risks of contamination.

The limitation of the volume of the liquefaction reactors relative to the SSF or SSCF or enzymatic hydrolysis reactors (the ratio of useful volume between the SSF or SSCF or enzymatic hydrolysis reactor and the liquefaction reactor is between for example 2 and 10) generally requires the implementation of a timing diagram in order to ensure the continuity of the different phases of operation (including the phases of filling, emptying, cleaning) between the two liquefaction and SSF or SSCF (or enzymatic hydrolysis) steps. This timing diagram is also established in order to limit the number of liquefaction and SSF or SSCF or enzymatic hydrolysis reactors by limiting downtimes.

### Embodiment According to the Invention

The description of the protocol according to the invention for the fed-batch liquefaction with an additional continuous step is shown diagrammatically with FIG. 2, which uses the same conventions as FIG. 1:

The durations of the phases indicated are examples.
A—Initial phase of filling of the liquefaction reactor 1

Preparation of the initial mixture in the liquefaction reactor: a certain amount of pretreated biomass 2 is mixed with a certain amount of water 3′ in order to achieve the desired solids content (SC). The stirring is started in order to homogenize the mixture and the pH and temperature is adjusted. The regulation of the pH is effected by addition of a basic solution 4, for example NH₄OH or KOH or NaOH if the pretreated substrate was produced under acidic conditions (for example impregnation with sulfuric acid and then steam explosion) or with the addition of an acidic solution if the pretreated substrate was produced under basic conditions.
B—starting of the fed-batch liquefaction-additions of pretreated biomass 2: once the initial mixture is suitably homogeneous, a certain amount of enzymes 3 (and of yeasts) is introduced into the reactor 1. This injection makes it possible to achieve the chosen dose of biocatalysts. After the injection of the biocatalysts, the fed-batch liquefaction starts: —after a certain time, the medium is already much less viscous and the first addition of pretreated biomass can take place. Depending on the strategy for adding the enzymes 3, additional enzymes may be added at this time. The mass of enzyme 3 introduced is proportional to the addition of pretreated biomass 2 to target a constant amount of enzymes/cellulose added. The additions follow one another until a mixture with the desired final mass is achieved. Generally, the mass and frequency of each addition are constant and regular.
F—Continuous phase of the liquefaction specific to the invention: a portion 5′ of the reaction medium is withdrawn and transferred to a downstream reactor (not shown) equipped with a standard stirrer. A portion of the inputs is added to the liquefaction reactor 1 in order to be at a constant volume: pretreated biomass 2, biocatalyst(s) 3, water 3′ and acidic or basic solution 4 for regulation of the pH. The continuous phase of the liquefaction is performed so that the rheology of the withdrawn medium remains less constraining or identical to that of the medium at the end of the fed-batch operation, so as not to negatively impact the operation of the enzymatic hydrolysis (or SSF or SSCF) reactor.

The operating conditions of the liquefaction in enzymatic-hydrolysis-only configuration (i.e. when targeting a conversion of the biomass into oligomeric or monomeric sugars) are preferably:
a temperature of between 25 and 80° C., preferably of between 4° and 60° C., and more preferentially still between 45° C. and 55° C.,
a pH of between 3 and 7, preferably of between 4 and 6 and more preferentially still of between 4.8 and 5.5.

The operating conditions of the liquefaction in simultaneous enzymatic hydrolysis and fermentation configuration (SSF or SSCF), i.e. when targeting the conversion of the biomass into alcohol by hydrolysis and fermentation, are preferably:
a temperature of between 25 and 80° C., preferably of between 3° and 50° C., and more preferentially still between 30° C. and 35° C.,
a pH of between 3 and 7, preferably of between 4 and 6 and more preferentially still of between 5.0 and 5.5.

The operating conditions (solids, dose of biocatalysts) are maintained at the target conditions, the parameters which make it possible to obtain the desired rheology are therefore the amount and the frequency of the additions and also the residence time of the marc in the liquefaction reactor over the course of the continuous phase. The residence time of the pretreated biomass in the liquefaction reactor during the continuous phase is greater than or equal to 4 h.

The duration of the continuous phase is for example between 1 hour and 170 hours, in particular between 10 hours and 72 hours.
C—Homogenization (optional): from 1 hour to 2 hours
D—Transfer of the reaction medium 5 from the reactor 1 to a downstream reactor (not shown); End of the liquefaction: after the continuous phase, all of the liquefaction medium is transferred to the enzymatic hydrolysis (or SSF or SSCF) reactor preferably equipped with a standard stirrer: duration between 2 hours and 4 hours
E—Cleaning of the liquefaction reactor 1. After transfer of the medium to the downstream reactor, the liquefaction reactor is cleaned to limit the risks of contamination: duration between 2 hours and 4 hours.

The enzymatic hydrolysis (or SSF or SSCF) is always performed in batch mode. The operating conditions of pH and temperature are identical to or different from the liquefaction. This is the case, for example, for a configuration in which the biocatalysts introduced at the liquefaction step are different from those introduced at the SSF or SSCF step.

The duration of the enzymatic hydrolysis (or SSF or SSCF) is between 10 hours and 170 hours, in particular between 70 hours and 140 hours.

According to a variant, only the enzymes are added to the liquefaction reactor 1, during the initial filling phase A and during the continuous phase F, and the yeasts are added to the SSF or SSCF reactor starting from the beginning of the continuous phase of liquefaction.

Of course, it is also possible to only perform an enzymatic hydrolysis, without yeast, or to utilize the sugars without transforming them into alcohol or by transforming them in a different way. It is also possible to perform the fermentation of the sugars separately, in a dedicated fermentation reactor.

Compared to the liquefaction according to the prior method (example in FIG. 1), the liquefaction according to the invention (example in FIG. 2) provides the following advantages:
Increase in the productivity of the liquefaction through a better utilization rate of each liquefaction reactor. The significance of the gain depends on the time of operation of this continuous phase, but also on the flow rate that it is possible to treat over the course of this phase (according to the residence time for the pretreated biomass during the continuous phase).
For the same amount of pretreated biomass to be liquefied, the number of liquefaction reactors is therefore reduced (or, for the same number of liquefaction reactors, the amount of liquefied biomass is greater): the liquefaction protocol according to the invention thus leads to a reduction in investment.
The phase of cleaning the liquefaction reactor is carried out after the transfer of several reactor volumes, which makes it possible to reduce the frequency of cleaning the reactors, and therefore to reduce the consumption of chemicals, and to increase the productivity by increasing the reaction time.
The ethanol production yields and the yields of conversion of the cellulose and hemicellulose into sugars are maintained at the same or virtually the same level.

### Example 1 (Comparative)

The protocol described above with the aid of FIG. 1 is used.

To illustrate the gain in the utilization rate of the liquefaction reactors, two timing diagrams will be compared: one for a known fed-batch liquefaction (FIG. 1), the other for a fed-batch liquefaction with a continuous phase (FIG. 2).

The liquefaction (+SSCF) is performed with a solids content SC of 20% by weight, the enzyme dose is 8 mg of enzymes/gSC, the amount of yeasts is 0.5 g/kg of medium.

The linearized flow rate of pretreated substrate feeding the liquefaction step is 15.6 tSC/h. The solids content of the pretreated substrate is 38% by weight.

Table 1 below details the timing diagram of the known liquefaction:

TABLE 1

| Operation | Duration | |
|---|---|---|
| Filling with water | 1 | h |
| Initial filling with pretreated | 2 | h |
| biomass in the liquefaction reactor | | |
| Addition of biocatalysts | 1 | h |
| Addition of pretreated biomass in | 9 | h |
| fed-batch mode to the liquefaction | | |
| reactor | | |
| Homogenization | 2 | h |
| Transfer to SSCF reactor | 2 | h |
| Cleaning | 3 | h |
| Total cycle time | 20 | h |

4 liquefaction reactors of 435 m³ (useful volume) are therefore necessary to liquefy the pretreated biomass with an operating utilization rate of 45%. The liquefied biomass is transferred into 4 SSCF reactors of 4500 m³ each, operating in batch mode.

The durations of the SSCF phases are indicated in the following table 2:

TABLE 2

| Operation | Duration | |
|---|---|---|
| Filling with liquefaction medium | 37 | h |
| Reaction | 113 | h |
| Transfer to SSCF reactor | 6 | h |
| Cleaning | 4 | h |
| Total cycle time | 160 | h |

### Example 2 (According to the Invention)

What follows is the liquefaction protocol according to the invention described above and illustrated in FIG. 2. Table 3 below details the timing diagram of the liquefaction according to the invention:

TABLE 3

| Operation | Duration | |
|---|---|---|
| Filling with water | 1 | h |
| Initial filling with pretreated biomass | 2 | h |
| in the liquefaction reactor | | |
| Addition of biocatalysts | 1 | h |
| Addition of pretreated biomass in | 8 | h |
| fed-batch mode to the liquefaction | | |
| reactor | | |
| Homogenization | 1 | h |
| Continuous phase in the liquefaction reactor | 20 | h |
| Homogenization | 2 | h |
| Transfer to SSCF reactor | 2 | h |
| Cleaning | 3 | h |
| Total cycle time | 40 | h |

2 liquefaction reactors of 500 m³ (useful volume) are therefore necessary to liquefy the pretreated biomass with an operating utilization rate of 72.5%. The residence time of the pretreated biomass in the liquefaction reactor is 8 h. The liquefied biomass is transferred to 4 SSCF reactors of 4500 m³ each, operating in batch mode.

The durations of the SSCF phases are the same as those indicated in table 2 for comparative example 1.

### Example 3 (Comparative)

Like comparative example 1, the known liquefaction protocol is used.

The liquefaction reactor is charged with a portion of the pretreated biomass, all of the water, the basic solution NH₄OH to adjust the pH to 5.3, and then all of the enzymes and yeasts are added; this corresponds to the time to of the liquefaction. The temperature is maintained at 33° C. The solids content in this initial mixture is 14% by weight. The fed-batch operation with pretreated biomass is then carried out with 12 additions of pretreated biomass for 6 hours: increase of 5% SC in 3 hours and then increase of 3% SC in 3 hours.

The solids content of the liquefaction medium is 22% by weight, the enzyme dose is 8 mg of enzymes/gSC, the amount of yeasts is 0.5 g/kg of medium.

The enzymatic solution has a protein concentration of 35 g/L and a density at 20° C. of 1.02 g/cm3.

The solids content of the pretreated biomass is 42% by weight.

The liquefaction reactor is equipped with a helical stirrer allowing good homogenization of the reaction medium and optimal management of the torque and viscosity.

The liquefaction medium is then homogenized for 2 hours to allow a reduction in stirrer torque, then transferred to an SSCF reactor in which the conversion of the cellulose and the hemicelluloses into sugars and the fermentation of the sugars into ethanol continue. The SSCF reactor is equipped with a conventional stirrer; this is, for example, a stirring system comprising 2 “TT” impellers with 3 blades (axial flow rotor) and a bottom turbine with two straight blades (radial flow rotor).

The duration of the liquefaction (initial filling+fed-batch operation with pretreated biomass+homogenization) is 12 hours. The duration of the SSCF is 132 hours (excluding liquefaction).

The operating parameters monitored are the torque of the stirrer in the liquefaction reactor, the contents of ethanol, glucose and xylose in the medium (analysis by HPLC) over the course of the SSCF reaction.

The performance of the SSCF process is evaluated using the following yields:
The ethanol/SC yield, equal to the ratio between the amount of ethanol produced and the total amount of solids introduced into the liquefaction reactor 1
From the total enzymatic hydrolysis on an end-of-fermentation sample: the yields of hydrolysis of the cellulose and xylan and the yield of ethanol production, calculated with respect to the Pasteur yield (i.e. 94.7% of the Gay-Lussac yield). As a reminder, the “Gay Lussac” yield is equal to the theoretical yield from the stoichiometric equation

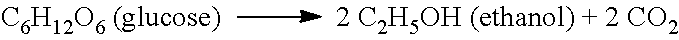

Which gives 51.1 kg of ethanol produced from 100 kg of glucose. Pasteur then demonstrated that there were co-products associated with the production of ethanol and CO₂ (notably glycerol, succinic acid, heavy alcohols, microorganism development). The Pasteur yield takes into account these sugar losses, i.e. 48.4 kg of ethanol produced from 100 kg of glucose. The Pasteur yield therefore corresponds to 94.7% of the theoretical yield. The results are indicated in table 4 below:

TABLE 4

| | Content of | Content of | | Yield of | Yield of | EtOH/ |
|---|---|---|---|---|---|---|
| | glucose at | ethanol at | EtOH/SC | hydrolysis of | hydrolysis | potential |
| | t_{final} | t_{final} | yield | cellulose | of xylan | EtOH |
| | g/kg | g/kg | g/100 g SC | % by weight | % by weight | % by weight |
| SSCF A | 1.15 | 56.0 | 25.4 | 81 | 90 | 79.7 |

### Example 4 (According to the Invention)

As for example 2, what follows is the liquefaction protocol according to the invention described above and illustrated in FIG. 2.

The phase of filling the liquefaction reactor up to the fed-batch phase of the substrate is identical to that of example 3. The continuous phase is then implemented: a portion of the medium is withdrawn and transferred to the SSCF reactor equipped with a standard vertical stirrer. A portion of the inputs (see table 5 below) is added to the liquefaction reactor for operation at constant volume.

The residence time of the pretreated biomass in the liquefaction reactor during the continuous phase is set at:
12 hours in the case where the biomass was pretreated under less acidic conditions (1.8% by weight of H₂SO₄ in the impregnation liquor)
6 hours in the case where the biomass was pretreated under more acidic conditions (2.4% by weight of H₂SO₄ in the impregnation liquor)

This residence time makes it possible to maintain a constant stirrer torque in the liquefaction reactor.

The duration of the continuous phase is 24 hours.

The amounts of medium to be withdrawn and also the amounts of inputs to be added to the liquefaction reactor, the useful volume of which is 3000 kg, are indicated in table 5 below. The nutrients are intended for yeasts (nitrogen source) and here are in the form of a solution of soluble corn proteins, in particular that sold under the name Solulys by the company Roquette.

TABLE 5

| | | | |
|---|---|---|---|
| Residence time in the liquefaction reactor | h | 6 | 12 |
| Amount of medium to be withdrawn | kg | 250 | 125 |
| Amount of water to be added | kg | 110 | 55 |
| Amount of enzyme solution to be added | kg | 13.2 | 6.6 |
| Amount of yeasts to be added | kg | 0.125 | 0.063 |
| Amount of nutrients to be added | kg | 0.145 | 0.073 |
| Amount of raw pretreated biomass to be added | kg | 125 | 63 |
| Amount of base solution (24% by weight | kg | 1.91 | 0.95 |
| NH₄OH) to be added | | | |

The operating parameters monitored are the torque of the stirrer in the liquefaction reactor, the contents of ethanol, glucose and xylose in the medium (analysis by HPLC, acronym for high-performance liquid chromatography) over the course of the SSCF reaction.

The performance of the SSCFs is evaluated as for example 3 above, the results are collated in table 6 below.

TABLE 6

| | Content of | Content of | | Yield of | Yield of | EtOH/ |
|---|---|---|---|---|---|---|
| | glucose at | ethanol at | EtOH/SC | hydrolysis of | hydrolysis | potential |
| | t_{final} | t_{final} | yield | cellulose | of xylan | EtOH |
| | g/kg | g/kg | g/100 g SC | % by weight | % by weight | % by weight |
| SSCF B | 0.96 | 58.0 | 26.4 | 85 | 100 | 82.1 |

It is thus verified that the yields, in particular those of ethanol, are identical, whether operation takes place with the liquefaction according to the known protocol (example 3) or according to the protocol of the invention (example 4).

The residence time makes it possible to maintain a constant stirrer torque in the liquefaction reactor.

The ethanol production and xylose consumption kinetics do not show any difference in reactivity between the SSCFs conducted with a standard conventional fed-batch liquefaction and the SSCFs conducted with a fed-batch and then continuous liquefaction according to the invention.

These good results in terms of performance can also be seen from the graph in FIG. 3. Specifically, curves C1 and C2 respectively correspond to the ethanol concentrations according to comparative example 3 and according to example 4 according to the invention: it can be seen that the ethanol concentrations coincide after 140 hours of SSCF. Similarly, curves C3 and C4 respectively correspond to the xylose concentrations according to comparative example 3 and according to example 4 according to the invention: here again, the falls in xylose concentration, as a result of the conversion thereof to ethanol, reach very low and similar concentrations after 140 hours. It can be seen that the invention also gives better results in terms of productivity in the first 48 hours.

1. A process for converting lignocellulosic biomass by comprising:
contacting, in an aqueous phase, pretreated lignocellulosic biomass with at least one biocatalyst (3) in a first reactor (1) containing a reaction medium comprising said pretreated lignocellulosic biomass (2) in the aqueous phase and said biocatalyst,
said process further comprising:
(a) a first step of liquefaction by addition of said pretreated lignocellulosic biomass and at least one biocatalyst to said reactor without withdrawing all or part of said reaction medium from said reactor, and
then (b) a second step of continuous liquefaction, with continuous withdrawal, from said first reactor, of a portion of said reaction medium, addition of at least one biocatalyst, and continuous addition of pretreated lignocellulosic biomass.

2. The process as claimed in claim 1, wherein, during the first liquefaction step (a), the additions of said pretreated lignocellulosic biomass (2) to the first reactor (1) are performed according to a fixed or variable frequency.

3. The process as claimed in claim 2, wherein, during the first liquefaction step (a), the additions of said pretreated lignocellulosic biomass (2) to the first reactor (1) are performed according to increasingly spaced-apart intervals of time.

4. The process as claimed in claim 1, wherein the second liquefaction step (b) is performed with a constant volume of the reaction medium contained in the first reactor (1).

5. The process as claimed in claim 1, wherein, during the second liquefaction step (b), pretreated lignocellulosic biomass (2) and at least one other compound, referred to as input, are added over time, and wherein at least one of the at least one other compound is chosen from: water (3′), an acidic compound, a basic compound (4), and a biocatalyst(s) (3).

6. The process as claimed in claim 1, wherein the rheology of the reaction medium is adjusted during the second liquefaction step (b) according to at least one operating condition selected from: the residence time of the pretreated lignocellulosic biomass (2) in the first reactor (1); and the amount and/or the frequency of additions of pretreated lignocellulosic biomass and input(s), wherein at least one of the input(s) is chosen from: water (3′), an acidic compound, a basic compound (4), and a biocatalyst(s) (3).

7. The process as claimed in claim 1, wherein the rheology of the reaction medium is adjusted during the second liquefaction step (b) so that it is identical to or less severe than the rheology of the reaction medium at the end of the first liquefaction step (a).

8. The process as claimed in claim 6, wherein the rheology of the reaction medium is monitored by monitoring the viscosity of the reaction medium, or the mechanical torque on a shaft of a stirring system fitted to the first reactor (1), or the electric power consumed by a motor driving for said stirring system.

9. The process as claimed in claim 1, wherein the first liquefaction step (a) has a duration of between 1 and 48 hours, in particular between 2 and 24 hours, and more preferentially still between 5 and 12 hours.

10. The process as claimed in claim 1, wherein the second liquefaction step (b) has a duration of between 1 and 170 hours.

11. The process as claimed in claim 1, wherein, during the second liquefaction step (b), a portion (5′) of the reaction medium is continuously withdrawn from the first reactor and sent to a second reactor in which, in a conversion step (c), the conversion of the biomass contained in the withdrawn reaction medium is continued in the presence of at least one biocatalyst.

12. The process as claimed in claim 1, wherein, at the end of the second liquefaction step (b), all of the reaction medium (5) is transferred from the first reactor (1) to a second reactor in which, in a conversion step (c), the conversion of the biomass contained in the transferred reaction medium is continued in the presence of at least one biocatalyst.

13. The process as claimed in claim 11, wherein step (c) in the second reactor is performed in fed-batch mode and then in batch mode, preferably for a duration of between 10 and 170 hours.

14. The process as claimed in claim 1, wherein said process comprises:
an initial step (a0) of filling the first reactor (1) with a first provision in said reactor of pretreated lignocellulosic biomass (2), biocatalyst(s) (3), water (3′) and optionally acidic and/or basic compounds (4),
a first step (a) of liquefaction in the first reactor (1) by addition of said pretreated lignocellulosic biomass (2) to said reactor without withdrawal, with the optional addition also of biocatalyst(s) (3),
then a second step (b) of continuous liquefaction in the first reactor (1), with continuous withdrawal, from the first reactor, of a portion (5′) of the reaction medium and transfer to a second reactor of said portion of the withdrawn reaction medium and addition of pretreated lignocellulosic biomass (2) over time,
then an optional step (b1) of homogenization of the reaction medium in the first reactor (1),
then a step (b2) of transfer of all of the reaction medium (5) from the first reactor (1) to the second reactor, in which conversion step (c) is performed preferably in batch mode,
and a step (b3) of cleaning the first reactor, in particular with the aid of an aqueous and preferably acidic or basic solution.

15. The process as claimed in claim 1, wherein the solids content SC of the pretreated lignocellulosic biomass used is at least 2% by weight and contains at least 10 g of cellulose per 100 g of solids.

16. The process as claimed in claim 2, wherein, during the first liquefaction step (a), the additions of said pretreated lignocellulosic biomass (2) to the first reactor (1) are performed in fixed amounts.

17. The process as claimed in claim 2, wherein, during the first liquefaction step (a), the additions of said pretreated lignocellulosic biomass (2) to the first reactor (1) are performed in variable amounts.

18. The process as claimed in claim 3, wherein, during the first liquefaction step (a), the additions of said pretreated lignocellulosic biomass (2) to the first reactor (1) are performed with fixed amounts of biomass.

19. The process as claimed in claim 9, wherein the first liquefaction step (a) has a duration of between 2 and 24 hours.

20. The process as claimed in claim 10, wherein the second liquefaction step (b) has a duration of between 10 and 72 hours.
